(19) United States
(12) Patent Application Publication
(10) Pub. No.: US 2025/0302429 A1
(43) Pub. Date: Oct. 2, 2025

(54) USING CONTRAST-ULTRASOUND TO QUANTIFY TOTAL CIRCULATING BLOOD VOLUME

(71) Applicant: QuantiPort, Inc., Portland, OR (US)

(72) Inventors: Jonathan Linder, Portland, OR (US); Lawrence Busse, Portland, OR (US)

(21) Appl. No.: 18/864,479

(22) PCT Filed: May 10, 2022
(86) PCT No.: PCT/US22/72233

§ 371 Date: Nov. 8, 2024

(51) Int. Cl. A61B 8/08; A61B 8/00; A61K 49/22

(52) U.S. Cl. CPC A61B 8/08; A61B 8/4488; A61B 8/481; A61K 49/223; A61B 8/4236

(57) ABSTRACT

The disclosed total blood volume determination systems and methods provide for a faster, simpler, more practical, less invasive, and more flexibly deployable device for blood volume measurement. These systems and methods are based on measuring, in a patient's bloodstream, the resonance of an agent injected into the circulating blood that allows for assessment of the agent's concentration and determination of the total blood volume. The agent may take a variety of forms, including an agent that is detectable via a non-invasive technique such as ultrasound. The agent may be measured over time and fitting the measurement data to provide an accurate determination of total blood volume.

### BACKGROUND

Determining circulating blood volume in human and veterinary patients, either once or repeatedly over the course of a treatment, is often important diagnostic information in both emergency medical care and long-term patient management. A method for rapid, accurate, and point-of-care determination of blood volume is not currently available. Measurement of blood volume provides vital diagnostic information that can guide therapy in particularly vulnerable populations, thereby greatly improving patient outcomes and decreasing health care costs by reducing unnecessary diagnostic and therapeutic interventions and reducing hospital length of stay.

Determining blood volume in human and veterinary subjects has been the subject of ongoing investigative work, but the current state of the art still has a range of disadvantages. A conventional method for determining blood volume involves injecting a subject with a radioactive indicator—specifically, human serum albumin (HSA) labelled with a radioactive iodine isotope such as iodine-131 (¹³¹I-HSA) or iodine-125 (¹²⁵I-HSA) and using a radiation sensor to measure the radioactivity in plasma obtained from a series of subsequent blood draws taken over 30-60 minutes. The plasma volume is determined and then hematocrit is used to deduce total blood volume. This technique involves several disadvantages. First, both exposing a patient to radioactivity and repeatedly drawing their blood may be detrimental to the patient's health, requires time which is a commodity in unstable patients; and administers radioactive substances to patients, which is contraindicated in patients such as children and pregnant women. Iodine-131 emits both beta and gamma radiation and is preferentially taken up by the thyroid gland and is known to potentially cause serious health side effects. This conventional technique also involves using specialized equipment, processes, and training for storing and deploying radioactive materials and the need to continuously reorder fresh radioactive materials and frequently dispose of unused radioactive materials. The equipment and storage facilities required are also voluminous, typically located in a specialized facility such as a nuclear medicine lab and are impractical for deploying in various medical scenarios such as in-situ emergency medicine in the field, or rural locations distant from any radio-oncology clinic or nuclear medicine facility.

Further, the conventional technique only measures plasma volume. Total blood volume must be inferred from plasma volume and hematocrit, so the accuracy of the measurement is contingent upon the accuracies of both plasma volume and hematocrit determinations. With this technique, a single measurement of total blood volume may take 1-3 hours. Blood volume changes rapidly and sometimes dramatically, so the results, once obtained, may no longer be valid or clinically relevant by the time the measurement is produced. Repeating blood volume measurements is complicated by the retention of the radioactive tracer in the blood system for 2-3 days after injection and the additive effects of additional IV injections of a radioactive material.

Further, any technique that relies on albumin is subject to inaccuracies. Albumin equilibrates with a non-circulating, marginal pool of plasma in the glycocalyx that lines all blood vessels, so the volume measured is the sum of the circulating plasma volume and non-circulating plasma volume. The non-circulating plasma component varies among individuals and is significant, roughly 1 liter (total blood volume (BV) is roughly 5 liters in adult humans) but is unpredictable; thus, a drawback of conventional techniques utilizing radiolabeled human serum albumin, indocyanine green, or other albumin-bound dyes is an overestimation of blood volume by about 15-25% due to the inclusion of non-circulating plasma within the glycocalyx matrix. Because plasma volume and hematocrit are used to calculate blood volume, the scale of the error in measuring plasma volume carries forward to the blood volume calculation. Albumin also readily leaks out of blood vessels when vascular permeability increases, which occurs in inflammation and shock and represents another source of significant error when albumin-based indicator methods are used in critical care patients. While the scale of the error of albumin-based techniques for determining blood volume is unknown, it is likely to be in the range of 15-40%, depending on an individual patient's non-circulating plasma volume and vascular permeability.

### SUMMARY

An embodiment provides a solution allowing for a faster, simpler, more practical, less invasive, and more flexibly deployable blood volume measurement. An embodiment provides a blood volume determining system that is based on measuring, in a subject's bloodstream, the resonance of an agent in the blood volume that allows for assessment of the agent's concentration and therefore determination of the total blood volume. The agent may take a variety of forms, for example an agent that is detectable via a non-invasive technique such as ultrasound. The agent may be measured over time, and fitting the measurement data, e.g., ultrasound data, over time via linear or nonlinear regression to a clearance model provides an accurate determination of total blood volume.

In one example, the measurement system accounts for initial rise in agent concentration during mixing with the bloodstream, a sustained peak circulating concentration, followed by a reduction of agent concentration due to clearing of the agent by the patient.

In one example, the agent may be a contrast agent such as solution containing microbubbles, nanobubbles or the like, similar to those which have a long history of inert, safe usage within human blood for contrast ultrasound imaging.

In one example, the system includes an ultrasound transducer and computational system that can measure the concentration of the agent in the bloodstream, such as by placement of a small ultrasound transducer over a blood vessel. This permits the system to operate without having to withdraw blood from the patient or subject.

In one example, the system or components thereof may be configured for a particular context, such as providing programmed routines or methods for automatically or semi-automatically determining blood volume in different clinical contexts or automatically or semi automatically recommending treatment options, such as drug dosing based on total blood volume determined for a patient.

In one example, an ultrasound transducer/receiver may be provided with an indicator light that guides the placement of the ultrasound transducer over a vessel by using Doppler ultrasound to detect blood flow. In another example, an ultrasound transducer/receiver array is placed over the skin of the arm, leg, or neck and the system determines which ultrasound element(s) within the array provides the best signal for analysis and selects this ultrasound element(s) for the analysis; such an arrangement may be used for example for field use where lighting or vessel-finding expertise may be lacking.

### BRIEF DESCRIPTION OF THE DRAWINGS

FIG. 1A illustrates agent concentration and ultrasound signal intensity relationship examples according to aspects of the disclosure.

FIG. 1B is an example of particle sizes and concentration of an example agent solution.

FIG. 2 is an example agent used in accordance with the disclosure.

FIG. 3 is an example system for using contrast-ultrasound to quantify total circulating blood volume in accordance with examples of the disclosure.

FIG. 4 is an example method of using contrast-ultrasound to quantify total circulating blood volume.

FIG. 5 illustrates a cross-section of a blood vessel showing distribution of traditional albumin-bound tracers or indicators and the disclosed contrast agent according to examples of the disclosure.

FIG. 6 shows a portion of a patient's arm with a transducer array positioned over the target vessel according to examples of the disclosure.

FIG. 7A shows the time-intensity curves for two nonhuman primate subjects after intravenous injection of 1×107 dose of tracer agent.

FIG. 7B shows the dose-intensity curves for two nonhuman primate subjects.

### DETAILED DESCRIPTION

Early identification of significant blood loss and the detection of ongoing blood loss are critical determinations in emergency care settings. The maintenance of blood volume is essential in providing profusion and organ support in order to reduce morbidity and mortality during the acute treatment and prolonged damage control resuscitation phases of blood loss associated with trauma as well as conditions such as gastrointestinal hemorrhage and post-partum hemorrhage. While critically important, objective and immediate blood volume determination is currently impossible in many emergency care environments. Physicians and emergency medical response personnel such as paramedics must rely upon surrogate parameters such as blood pressure, hematocrit, and heart rate. These surrogate methods have been proven to be inaccurate or even misleading, resulting in preventable morbidity and mortality. For example, young, healthy people often have robust neurovascular compensatory responses to near-lethal blood loss that maintains normal blood pressure in spite of significant blood loss. These patients may not be recognized by hospital personnel as emergent until physiological decompensation and hemorrhagic shock that require immediate and aggressive resuscitation efforts to prevent mortality. Conversely, a patient treated for severe trauma may have low hematocrit after resuscitation therapy. The low hematocrit could be mistakenly interpreted as ongoing blood loss when the low hematocrit is, in fact, the result of hemodilution from prior aggressive fluid therapy.

Objective blood volume determination is a promising diagnostic tool that could accurately direct resuscitation and triage decision-making as well as aid in judicious use of fluids and blood transfusions in austere environments. As disclosed herein, embodiments have been developed that provide novel devices, systems, and methods for the in vivo determination of blood volume. The disclosed devices developed are small and durable enough to be used in the field, at a point-of-care, pre-hospital, and hospital settings to improve diagnostic accuracy and guide therapeutic decision-making.

The only commercially available device for blood volume measurement is the Blood Volume Analyzer, BVA-100 (Daxor® Corporation, New York, NY). The BVA-100 is a semiautomated system that uses radiolabeled human serum albumin (¹³¹I-HSA) as a blood volume indicator. Daxor® supplies this blood volume indicator in a dose syringe, marketed as Volumex®. A known quantity of ¹³¹I-HSA is injected into the circulation of the patient. After the blood volume indicator has mixed fully throughout the patient's circulatory system, a series of five consecutive blood samples are withdrawn at fixed time intervals. The known dose of radioactivity equilibrates in plasma, and the subsequent degree of dilution is directly proportional to the volume of the diluent. Radiation measurements from the plasma of the five consecutive blood draws allow a linear extrapolation of the radioactivity concentrations to time zero, resulting in a plasma volume determination. The plasma volume and hematocrit (obtained from a centrifuged blood sample) are then used to deduce the total blood volume. A significant drawback of this method is that the radioactive isotope requires special licensing and handling, and exposure to personnel and patients presents risk and side effects. Exposure to radioactivity may also become an obstacle for patient consent. The diagnostic test and analysis are prolonged, often taking 4-6 hours before results are available in some settings.

In many cases, even with semi-automating the laboratory processing steps, the clinical relevance of the results using the Daxor® BVA-100 technique has long passed by the time these results are available. Additionally, the ¹³¹I-HSA remains in circulation for 18-28 hours, which complicates any repeat measurements during this period. Additional obstacles are the high purchase price of the Daxor® machine cluster as well as the annual maintenance fees and the extensive laboratory space required to house and operate these machines. Because the indicator is radioactive, the BVA machines are typically housed within a nuclear medicine laboratory, which is not available in most hospitals, emergency care settings, or in under-resourced locations. Furthermore, recent studies have indicated a bias towards overestimation of blood volume when HSA is used as a blood volume indicator for these measurements. The likely reason is HSA equilibrates with circulating plasma and non-circulating plasma within the glycocalyx matrix that lines blood vessels. Additionally, HSA binds to albumin receptors on the endothelial cells lining the blood vessels and readily leaks out of circulation and into the interstitial plasma pool, resulting in false dilution of the blood volume indicator and a profound overestimation of blood volume. Given all of the problems with current methods of measuring blood volume, additional methods, systems, and devices are needed.

To meet the needs identified above, which represents a non-exhaustive list, disclosed herein, by way of example, is a blood volume analyzer system, as well as related methods and products, which uses an ultrasound transducer that is positioned onto the patient's skin to measure the resonance of an injected agent such as a microbubble or nanobubble contrast agent. As disclosed herein, a device for determining total blood volume is small, lightweight, and rugged, meeting the needs of a clinical or field-ready device.

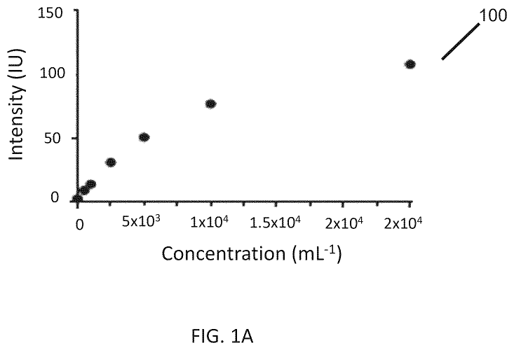

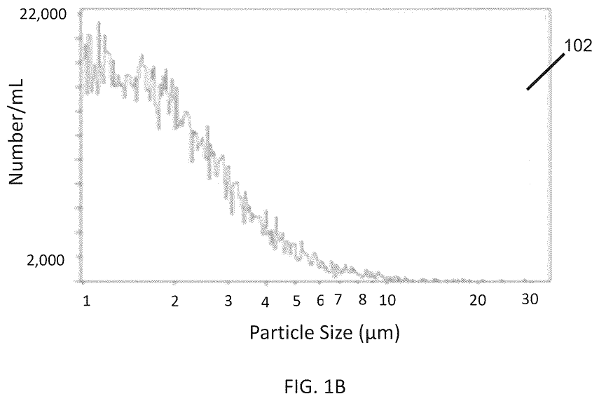

In one example, total blood volume is calculated by measuring the ultrasound signal strength of an agent that is injected into the circulatory system. The agent is specifically formulated to have a stable concentration in circulation for a period of time, creating a plateau concentration when plotted against time before the agent concentration decreases with time as it is metabolized or otherwise eliminated from circulation. Microbubble or nanobubble contrast agents resonate in an ultrasound beam, rapidly contracting and expanding in response to the pressure changes in a sound wave. Ultrasound transducers can detect this resonance, or signal. The signal intensity is linearly (or slightly non-linearly) directly related to agent concentration (at the dose of agent administered for this application, FIG. 1A) and therefore concentration in circulating blood can be deduced from the signal intensity at plateau. The circulating blood volume equals amount of injected contrast agent (volume injected×concentration) divided by the concentration of contrast agent in blood circulation.

In one example, a concentration or dilution methodology is used to calculate circulating blood volume. The signal intensity (rather than narrow point peak) reaches a sustained peak during the plateau period and is the result of specific microbubble or nanobubble agent design and construction. The sustained peak of signal intensity allows for a mathematically simplified approach to the conventional technique to measure blood volume in that it does not involve curve fitting regressions, which have inherent flaws associated with statistical inference and extrapolation. The peak signal intensity may be corrected for attenuation based on the depth of the vascular flow analyzed. However, the use of low concentrations of microbubble or nanobubble contrast agent with super-resolution processing eliminates the need for such correction. Moreover, at the relatively low ultrasound frequency used (approximately 5 MHz) and shallow vascular depth in the upper arm or neck, the attenuation will be almost negligible (less than 2 db difference between individual patients). The resulting peak signal intensity is converted to an agent concentration as follows. As shown in FIG. 1A, the backscattered ultrasound signal intensity from agent resonance, such as a contrast agent microbubble (MB) or nanobubble (NB), has a linear relationship to the concentration of the MB or NB agent in circulation over a wide range of concentrations 100. The signal intensity is generated by stable cavitation (volumetric oscillation of the agent in the acoustic field) of the nanobubbles as they flow through blood. The signal intensity is then matched or compared against the concentration v. signal intensity standard to determine circulating concentration. The standard can be established from prior measurements made either in vitro (using phantoms) or in vivo (using an animal model). FIG. 1A illustrates concentration and ultrasound signal intensity relationships 100 obtained at 7 MHz for an example agent. This allows the determination of agent concentration within circulation at equilibrium. Once this concentration has been calculated, the total blood volume may be determined by solving for V₂ in the following equation: C₁V₁=C₂V₂; where C₁ is equal to the concentration of agent injected; V₁ is equal to the volume of agent injected; C₂ is equal to the measured and calculated concentration of agent after injection; and V₂ is equal to the total circulating blood volume.

In one example, fitting a time course of measurements for the concentration of the agent to a model of the concentration of the agent decreasing as a function of time includes using regression to fit the time course for the concentration of agent.

As shown in FIG. 1B, an example of particle sizes and concentration of an example agent solution 102 is illustrated. As shown, the particle sizes range from 1 to 10 micrometers, with most of the particles being in the 1 micrometer to 2 micrometer size range (microbubbles). In another example, nanometer particle sizes (nanobubbles) may be used as the agent, i.e., specifically less than 1 micrometer in diameter.

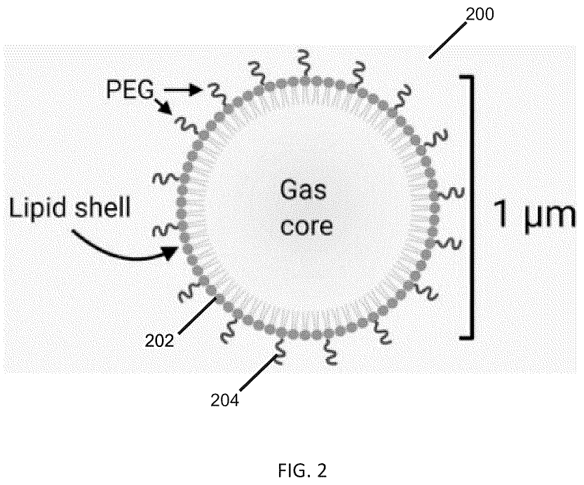

Illustrated in FIG. 2 is an example particle 200 for use as an agent for determining total blood volume. In FIG. 2 the example particle 200 is formed with a distearoylphosphatidylcholine (DSCP) lipid shell 202 with polyethylene glycol (PEG)-stearate that encloses a heavy gas (decafluorobutane) core 204. As indicated in the example of FIG. 2, the particle size is about 1 micrometer.

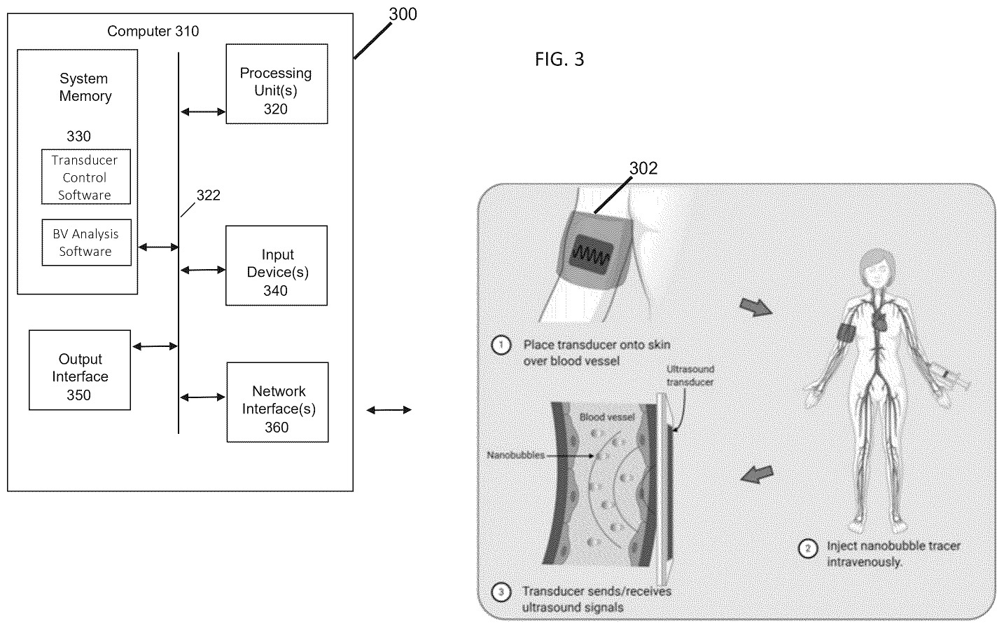

In an example system 300, the dilution and elimination of the agent in circulating blood is followed over a time course. The system 302 may include an ultrasound transducer that can be integrated with a standard blood pressure cuff, as described herein and illustrated in FIG. 3. The dilution of the agent to its concentration at stable cavitation may be directly proportional to total diluent (blood) volume. Thus, being able to determine the degree to which the agent is diluted allows for the determination of the total blood volume of a subject based on the equation described above.

In the example shown in FIG. 3, a small ultrasound transducer is placed in association with, e.g., reversibly attached to, embedded, etc., an arm band such as a blood pressure cuff. The arm band is placed onto the arm of the patient. The agent is injected via syringe intravenously. Alternatively, the transducer may be secured to the skin over a blood vessel using adhesive backing as shown in FIG. 3 or held in place over the blood vessel by any suitable means.

The ultrasound data needed to determine BV does not require the production of images, i.e., a non-imaging ultrasound transducer is suitable for this application. The transducer detects the concentration of circulating agent according to a predetermined protocol, e.g., programmed into the memory of the ultrasound system, or a device communicatively coupled to the transducer. In one example, the predetermined protocol obtains measurements several times per second. The concentration change over time is used to determine the elimination rate of the agent or the rate at which the agent is removed from circulation, typically by the patient's liver. Using the elimination rate, a regression analysis of the concentration over time can then be used to calculate the concentration value if the agent were to be perfectly mixed or distributed in the blood and prior to any removal. This agent concentration value is then used to calculate circulating total blood volume based on a conservation of matter equation, as described herein.

If the time-signal intensity graph shows the signal intensity maintains linearity for a long enough period of time to make the volume calculation, no slope to regression is needed. The sustained peak signal intensity and correlating agent concentration is converted to volume using an agent concentration-to-volume standard, as discussed above.

In certain embodiments, the device, system, and methods use ultrasound contrast agent in the form of microbubbles or nanobubbles as the agent. Ultrasound contrast agent mixes thoroughly and completely within the circulating compartment, and then is eliminated from the patient's system, typically within 15-20 minutes. Furthermore, microbubble and nanobubble ultrasound contrast agents have been used for medical imaging applications for many years and are considered safe, even in pregnant women and children. The agent referenced herein may be used in any pharmaceutically acceptable formulation, medium, or carrier including, but not limited to, lyophilized, micellular, microbubble, nanobubble, lipid, protein and liposomal formulations.

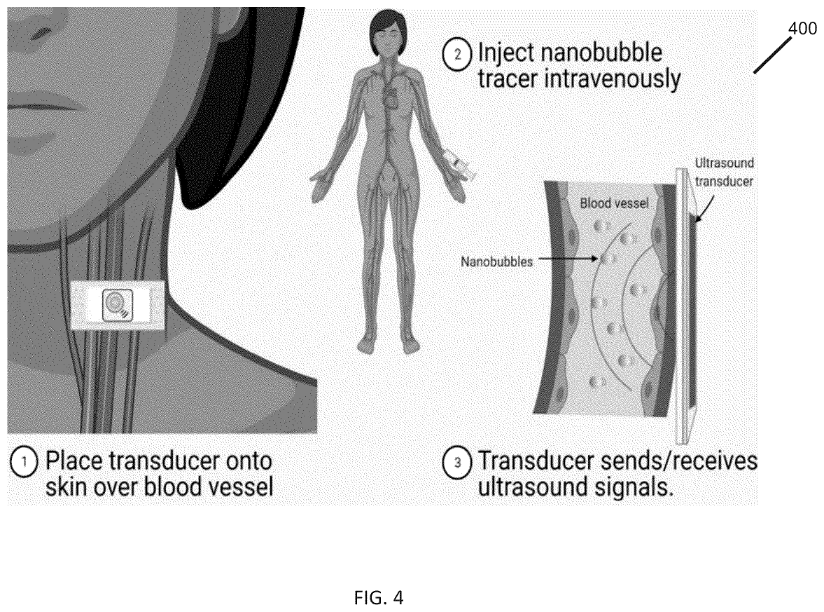

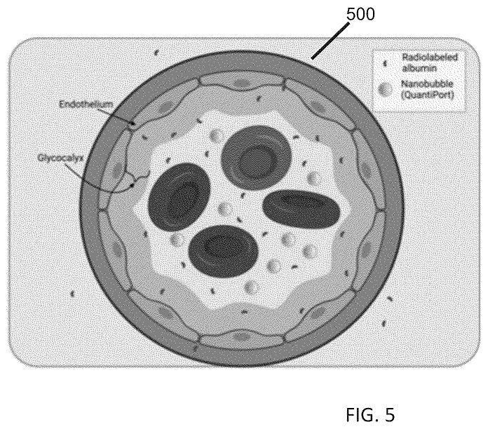

In some examples, the use of a lipid-stabilized microbubble or nanobubble ultrasound contrast agent is preferred to albumin-bound agents because these micro-or nanobubble agents do not enter, mix or associate with extra-circulatory plasma volumes 400, as illustrated in FIG. 4. Shown in FIG. 5 is a cross-section of a patient's blood vessel 500 indicating the distribution of traditional albumin-bound tracers or indicators that readily equilibrate with the non-circulating plasma pool within the proteoglycan matrix of the glycocalyx as well as the interstitial plasma pool, resulting in an overestimation of circulating blood volume, for example ranging from 15-25% error as described herein. Whereas a microbubble or nanobubble contrast agent, as described herein, is roughly 200 times larger in size than albumin and is surrounded by a lipid, protein, or biopolymer shell, resulting in an agent with similar rheology to a red blood cell. These characteristics, and others, ensure the agent stays within the circulatory pool and is unaffected by changes in vascular permeability, which enables far more accurate total blood volume determination without being confounded by vascular permeability changes.

Embodiments provide methods for determining total blood volume in a variety of contexts. In some examples, one or more pre-injection measurements may be taken, e.g., as a baseline, or background resonance signal. In an example, detecting the magnitude of the agent's signal in ultrasound data over time includes acquiring background followed by a set of two or more post-injection measurements of the agent over a time period, e.g., at a predetermined interval, manually, or in response to a sensed environmental or vital sign parameter. The method may further include determining a post injection computed agent value for each of the post-injection measurements. In some examples, determining the post-injection computed agent value employs a model that simulates the post-injection agent amount, for example, applying a regression model to arrive at the post-injection agent amount.

An embodiment employs a non-radioactive agent with several months' shelf life. The agent is injected intravenously and is eliminated by the liver and reticuloendothelial system within about 15 minutes. The agent stays within the circulating blood pool regardless of vascular permeability changes and does not equilibrate with plasma in the glycocalyx. Further, an embodiment does not require blood sampling or laboratory processing of blood samples.

Referring back to FIG. 3, an example system 300 that may be used in implementing an embodiment includes one or more of an ultrasound transducer, which may take the form of a non-imaging ultrasound transducer that is formed as a small, durable pad that can be positioned next to the skin. In one example, the ultrasound transducer may be used as a stand-alone pad or patch positioned over a vein or artery. In another example, the ultrasound transducer may be a component of a modified blood pressure cuff, for example as a component of the inner lining of the cuff. A computational program, for example associated with transducer or an associated device, performs a program or routine to obtain signal intensity measurement data related to the concentration of the intravenously injected tracer agent. The overall time to obtain an agent concentration useful in a total blood volume measurement result may be 2-3 minutes. The program or routine may be repeated, with or without modifications from the first program run, e.g., repeated every 15-20 minutes to obtain additional blood volume measurements, e.g., for use in determining rate of ongoing blood loss or for use in determining the effects of therapeutic interventions such as blood transfusion.

In some embodiments, the program or routine may be customized or arranged for a given context, for example programmed to run with minimal user input for use in a before and after context, such as determining total blood volume before and after an event, such as a major surgery, labor/childbirth, a blood transfusion, or the like. Further, a program may be configured to intermittently determine agent concentrations for different automated calculations, such as determining a bleeding rate from internal injury or gastrointestinal (GI) bleed, producing automated control signals for external systems, e.g., drug dosing systems, etc.

The example methods of use provided herein relate to use of an agent with similar rheology to red blood cells, which is large enough in size (0.8 to 2 micron diameter) to stay within the circulating blood pool and does not equilibrate with the interstitial space or the marginal non-circulating plasma in the glycocalyx. All conventional albumin-based tracer agents (including radio-iodinated albumin methods considered to be the gold standard, as well as ICG methods as ICG immediately binds to albumin upon IV injection) equilibrate with the plasma in the glycocalyx, which is variable among individuals and may also vary within an individual depending on their hydration status and systemic inflammation level. Plasma within the glycocalyx is estimated to be approximately 1 Liter. Total blood volume of a human is approximately 5 L, so adding 1 L is a 20% overestimation of plasma volume. Because these methods use plasma volume and hematocrit to deduce total blood volume, the error is mathematically compounded in the total blood volume calculation. Furthermore, the albumin-bound tracers readily leave the circulating pool whenever vascular permeability increases, as occurs in inflammation and shock. The result of extravasation of the tracer is additional overestimation of plasma volume and therefore blood volume. By using a microbubble or nanobubble agent, the agent stays within blood vessels, does not equilibrate with the plasma in the glycocalyx, and remains in the circulating pool regardless of vascular permeability changes. Therefore, the result is more clinically relevant and far more accurate (by eliminating the 15-40% error discussed herein), especially in shock and other critical care patients.

The example methods described herein are also less invasive than use of blood draws, which need to be repeated, or use of an inserted probe, e.g., an IV optical fiber probe, as the example methods described herein do not require intravenous placement of the detector or measurement device. Rather, in an embodiment, the detector or measurement device is an ultrasound transducer placed on the skin. The agent is not albumin-bound, and only one IV catheter is required for the injection of the agent. The agent is a derivative of microbubble-based ultrasound-enhancing agents that are currently used in millions of patients per year in cardiac and abdominal ultrasound studies. This technology has been proven safe, even in critically ill patients, and its accuracy is unaffected by vascular permeability changes that occur in inflammation and shock.

In an embodiment, an agent will be just under 2 micrometers in diameter, whereas current conventional commercially-available microbubbles range in size 2-4 micrometers in diameter. In an embodiment, use of a smaller size agent, e.g., less than 2 micrometers, with neutral charge and PEG increases circulation half-life and therefore provides a longer plateau of ultrasound signal intensity during which measurements can be taken. In an embodiment, the agent may also be designed to produce a signal-to-noise ratio so that signal saturation does not occur at low concentrations, thus enabling the quantification of subtle changes in circulating concentration. To modify resonant signal, the size of the bubble agent used may be reduced and/or the constitution of the bubble shell may be altered. In an embodiment, a nanobubble agent is 0.8 to 2 micrometer in diameter, with low surface charge, highly PEGylated, and lipid or polymeric (PLGA) shell. The nanobubble agent core comprises a high molecular weight gas with low solubility and low diffusivity, such as a decafluorobutane, as illustrated in FIG. 2. Such agents may be formulated so that they do not require refrigeration and, in a lyophilized form, the shelf live is months to years.

Further, the small diameter nanobubble agent has a narrow size distribution meaning that the variation of the agent diameters in any given batch vary, but within a relatively small size range, such as a mean diameter of 900 nm, with standard deviation (S.D.) less than 200 nm. Any suitable size and distribution can be used. As explained above, there is a linear correlation between the ultrasound signal intensity and the micro-or nanobubble agent concentration upon which the calculation of total blood volume relies. FIG. 1B shows an example narrow size distribution of the microbubbles within the batch ranging from 1.0-2.0 microns in diameter 102. With a smaller variation of the contrast agent size, the accuracy in determining their concentration based on signal intensity increases, and therefore improves blood volume measurement accuracy. The relatively small size of the contrast agent (around 1 micron) will generate less resonant signal and will also cause less attenuation due to the lower extinction coefficient, resulting in the concentration-intensity signal staying linear for a longer time which improves the accuracy of the regression.

When the contrast agent size variability increases, the resonant ultrasound signal variations result in inaccurate concentration determinations. When the contrast agent size variability decreases, the resonant ultrasound signal intensity can be used to accurately determine contrast agent dilution in order to calculate blood volume, according to the known proportionality of signal intensity to concentration.

Many commercially-available microbubble contrast agents for applications like cardiac ultrasound, for example, have a diameter size range that varies between 1-4 microns. This size variation does not allow for accurate quantification of contrast agent in the blood due to the range of resonant signals produced.

The contrast agent size and distribution could vary depending on certain characteristics of the contrast agent, such as the type of inert gas core and the characteristics of the shell surrounding the core, for example. Further, the agent size and distribution could also vary based on the required sensitivity and specificity of a diagnostic result in various applications.

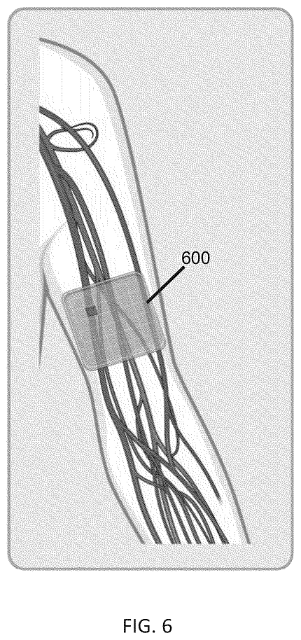

In an embodiment as shown in FIG. 6, multiple (e.g., 16-25) non-imaging transducers are arranged into a transducer array 600 that is placed onto the surface of the skin, directly over a vessel, typically on the arm or neck. The device identifies the transducer within the transducer array that has the highest contrast agent signal-to-noise ratio where noise is considered to be tissue signal, and then selects this transducer for the analysis. This process of identifying the best available transducer signal from the array of transducers by evaluating or comparing various signal characteristics (i.e., Doppler signal intensity) eliminates the need for the operator to manually locate a blood vessel. In some examples, the signal from a single transducer is used and in other examples multiple signals from different transducers producing quality signals can be used to validate the signal data and ultimately the calculations of blood volume. The quality signals can be identified by a preset criteria of signal characteristic(s). While blood vessel localization function may use Doppler, the contrast agent detection will be via multi-pulse algorithm at low mechanical index. This algorithm detects the linear and a small amount of non-linear signal from tissue at baseline, and then detect the non-linear additional signal contribution from the contrast agent after injection. The functionality for changing the selected transducer if there is patient movement will be included as a fail-safe.

Using two or more frequencies in the proposed application allows for corrections to the received Doppler signal. For example, the use of several, appropriately chosen, transmitted frequencies allow one to discern the distribution of the bubble sizes in the contrast agent. Understanding the distribution of bubble sizes allows one to correct for any manufacturing variation in bubble sizes in the proposed contrast agent. The resonant frequency varies with different sized bubbles—the bubbles “ring” or resonate differently depending on their size. Bubbles with a specific size and specific resonance characteristics produce a desired resonance profile. Such a targeted resonance profile can help the algorithm detect bubbles outside of the targeted resonance range and adjust the range of analysis, if necessary.

Other frequencies can be chosen that are well outside the resonant frequency of the contrast agent, which provides a real-time Doppler signal from the other acoustic scattering components of the blood. This, in turn, provides a real-time base line reference signal that characterizes the size, depth, and pulsatile flow of the vascular segment being analyzed for inclusion into the algorithm that calculates blood volume.

A signal analysis can be performed in one or multiple transducers to determine if it detects blood flow before performing further ultrasound signal analysis. The signal analysis algorithm determines whether signals from a transducer are produced by blood flow, confirming that the transducer is physically placed over a vessel. Alternatively, several transducers could be arranged into an array, such as the example patch shown in FIG. 6, and the transducer array placed onto an anatomical region in which patients have target vessels. The multiple transducer signals are evaluated to see which is best based on a comparison between signals and/or a comparison to empirical or threshold data regarding the expected signal characteristics for the target vessel. When the best intensity signal (or signals) is found, the transducer(s) is selected for the analysis. The remaining unselected transducers will continue to collect data for redundancy as a failsafe against transducer misalignment due to patient movement. Doppler recognition properties, including phasic changes in intensity, vary depending on whether the blood flow analyzed is arterial or venous.

When the target vessel is found, a visual, tactile, audible or other indicator may be output for the caregiver that indicates the transducer, in whatever form they are placed on the patient, is properly positioned. The user feedback or indicator could be a green light that illuminates when a quality ultrasound (Doppler, or otherwise) signal is found, for example. Any suitable user feedback or a combination of feedback could be used.

In an embodiment, the transducer may be incorporated into the inner lining of a blood pressure cuff, resulting in a device that measures blood volume as well as additional parameters, e.g., heart rate and blood pressure. In an embodiment, the ultrasound system will detect blood flow and automatically select the transducer cell within an array of transducers that yields the optimal signal for analysis.

In an embodiment, a method includes injecting a known quantity of agent intravenously. The concentration of injected agent is measured several times per second within the blood vessel via the transducer. As the agent is removed from circulation (e.g., by the liver), an elimination curve is generated by software running on a programmable device, or a device communicatively coupled to the transducer. This line or curve will be used to back-extrapolate the agent concentration to the time of first signal capture to then calculate the total circulating blood volume. Other possible configurations of the transducer include a small, non-flexible transducer with a Doppler flow detector to aid in positioning over a blood vessel such as a vein. In an embodiment, the ultrasound transducer performs well over either a vein or an artery.

An embodiment may be used in a variety of contexts. For example, an embodiment may be used in a trauma context, where the trauma results in hemorrhage (internal or external), for GI bleeds, before and after major surgery to determine blood loss, before and after childbirth, burn victim fluid management, kidney failure management, congestive heart failure management.

In an embodiment, the system or components thereof may be specifically programmed to assist with various contexts. By way of example, in an oncology context, a program may be designed to determine a total blood volume for a patient, e.g., a pediatric oncology patient. Further, a program may automatically determine a chemotherapeutic drug dosing based on the determined total blood volume. By way of specific example, in children safe doses of oncology medication are generally unknown and based on historic doses given to similar cases.

Ultimately, a dose should be delivered that is effective in treating cancer but not toxic. In an embodiment, dosing is guided by the desired blood concentration of the medication, which is derived automatically from the total blood volume determination. Such determinations may be output automatically as part of a program or software module that is configured for a given context, e.g., pediatric oncology. In some examples, the output data may be used to display values, recommendations, or the like to a caregiver. In some examples, the output data may be used to generate or as a control signal to an external system, such as a drug dosing system that delivers medication via intravenous drip or dispenses medication based on blood volume determination(s).

In one example, illustrated in FIG. 6, an ultrasound transducer in an ultrasound transducer array may be provided with an indicator light that guides the placement of the ultrasound transducer over a vessel. In certain embodiments, an ultrasound transducer array is placed over the skin of the arm, leg, or neck, illustrated in FIG. 6, and the automated system determines which ultrasound transducer within the array provides the best signal for analysis and then selects this ultrasound transducer for the blood volume analysis; such an arrangement may be used for example for field use where lighting or vessel-finding expertise may be lacking.

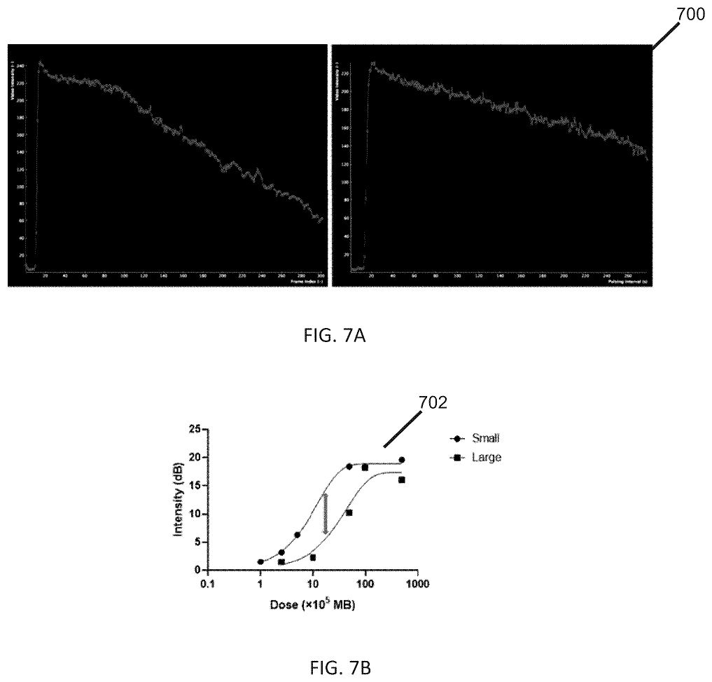

FIGS. 7A and 7B show preliminary data from two Japanese macaques that employed the disclosed methods and systems of quantifying total blood volume using contrast-ultrasound. In these “dose-response” experiments, MBs were injected into a peripheral vein and an ultrasound system was used to produce time-intensity curves of the non-linear fundamental component (amplitude modulation imaging at 6 MHz, mechanical index 0.14, dynamic range 55 dB) of intravascular MBs within the jugular vein. Examples of two time-intensity curves 700 are shown in FIG. 7A. Both subjects received a 1×107 dose of tracer agent intravenously. The “Small” subject (left panel) had a calculated blood volume of 376 mL while the “Large” subject (right panel) had a calculated blood volume of 550 mL. The decay to near background level occurred in 15 minutes, which allows repeat measures in as little as 20 minutes. FIG. 7B shows the dose-intensity relationships 702 for measurements taken at 40 seconds post-appearance demonstrated relatively long, linear segments indicating robust differences in signal according to BV can be achieved over a wide range of MB doses in these animals, from 1×106 to 1×107. The signal intensity differences (approximately 2-fold difference) in the two subjects accurately reflected calculated differences in the total BV of the subjects based on equations validated for normal rhesus macaques.

It will be readily understood that certain embodiments can be implemented using any of a wide variety of devices or combinations of devices. Referring back to FIG. 3, an example device that may be used in implementing one or more embodiments includes a computing device (computer) 310, for example included in a transducer or device communicatively coupled to the transducer (e.g., a laptop or desktop computer, an in-room medical device, a tablet computing device, etc.).

The computer 310 may execute program instructions or code configured to store and process data (e.g., ultrasound data from a transducer, control function data for controlling a transducer to obtain measurements, etc., as described herein) and perform other functionality of the embodiments. Components of computer 310 may include, but are not limited to, a processing unit 320, a system memory 330, and a system bus 322 that communicatively couples various system components including the system memory 330 to the processing unit 320. The computer 310 may include or have access to a variety of non-transitory computer readable media. The system memory 330 may include non-transitory computer readable storage media in the form of volatile and/or nonvolatile memory devices such as read only memory (ROM) and/or random access memory (RAM). By way of example, and not limitation, system memory 330 may also include an operating system, application programs, other program modules, and program data. For example, system memory 330 may include application programs such as transducer control software, other control software, and/or ultrasound data blood volume (BV) analysis software. Data may be transmitted by wired or wireless communication, e.g., to or from a transducer or a local computing device to another computing device, e.g., a remote device or system.

A user can interface with (for example, enter commands and information) the computer 310 through input devices 340 such as a touch screen, keypad, etc. A monitor or other type of display screen or device can also be connected to the system bus 322 via an interface, such as an interface 350. The computer 310 may operate in a networked or distributed environment using logical or physical connections, e.g., facilitated by network interface(s) 360, to one or more other computers or databases. The logical connections may include a network, such local area network (LAN) or a wide area network (WAN) but may also include other networks/buses.

It should be noted that the various functions or method steps described herein may be implemented using processor executable instructions stored on a non-transitory storage medium or device. A non-transitory storage device may be, for example, an electronic, electromagnetic, or semiconductor system, apparatus, or device, or any suitable combination of the foregoing. More specific examples of a non-transitory storage medium include the following: a portable computer diskette, a hard disk, a random-access memory (RAM), a read-only memory (ROM), an erasable programmable read-only memory (EPROM or Flash memory), a portable compact disc read-only memory (CD-ROM), solid state memories, or any suitable combination of the foregoing. In the context of this document “non-transitory” includes all media except non-statutory signal media.

Program code embodied on a non-transitory storage medium may be transmitted using any appropriate medium, including but not limited to wireless, wireline, optical fiber cable, RF, etc., or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), a personal area network (PAN), or the connection may be made through other devices (for example, through the Internet using an Internet Service Provider), through wireless connections, or through a hard wire connection, such as over a USB connection.

1. A system to quantify total blood volume, comprising:
an ultrasound transducer configured to:
receive a resonance signal representative of detected resonance of a contrast agent injected into a patient bloodstream;
determine a circulating concentration of the agent in the patient bloodstream based on a characteristic of the resonance signal;
quantify an agent concentration in the patient bloodstream based on the characteristic of the resonance signal; and
an output configured to output the quantified agent concentration.

2. The system of claim 1, wherein the resonance signal includes an initial agent concentration portion, a sustained peak agent concentration portion, and a clearing agent concentration portion, and wherein the ultrasound transducer is further configured to determine the circulating concentration of the agent in the patient bloodstream based on a characteristic of the sustained peak agent concentration portion of the resonance signal.

3. The system of claim 1, wherein the resonance signal further includes a pre-injection portion and a post-injection portion of the resonance signal, the pre-injection portion correlating to a time before the agent is injected into the patient bloodstream and the post-injection portion correlating to a time after the agent is injected into the patient bloodstream, and wherein the ultrasound transducer is further configured to compare the characteristic of the of the resonance signal during the pre-injection portion to the characteristic of the resonance signal in the post-injection portion.

4. The system of claim 1, wherein the characteristic of the resonance signal includes an amplitude of the resonance signal.

5. The system of claim 1, wherein the ultrasound transducer is further configured to determine the circulating concentration of the agent in the patient bloodstream based on the characteristic of the resonance signal taken at multiple times after the agent is injected into the patient bloodstream.

6. The system of claim 1, wherein the ultrasound transducer is further configured to transmit an instruction to cause an ultrasound transducer to measure the resonance signal of the agent in the patient bloodstream.

7. The system of claim 1, wherein the ultrasound transducer is further configured to generate a treatment recommendation based on the quantified agent concentration and the output is further configured to output the treatment recommendation.

8. The system of claim 1, wherein the ultrasound transducer is further configured to:
determine the circulating concentration of the agent in the patient bloodstream based on the circulating concentration of the agent in the patient bloodstream and an elimination rate of the agent in the patient bloodstream; and
quantify the agent concentration in the patient bloodstream based on the characteristic of the resonance signal and the elimination rate of the agent in the patient bloodstream.

9. The system of claim 1, wherein the agent includes a microbubble or a nanobubble.

10. The system of claim 9, wherein the microbubble or the nanobubble agent has a diameter of 4 micrometers or less.

11. The system of claim 10, wherein the microbubble or the nanobubble agent remains within the target plasma pool of the patient bloodstream from a time the agent is injected into the patient bloodstream until the agent is eliminated from the patient bloodstream.

12. The system of claim 1, wherein the ultrasound transducer is further configured to:
identify a stable concentration portion of the resonance signal, the stable connection portion representative of a plateau concentration of the agent in the patient bloodstream over a time period; and
determine the circulating concentration of the agent in the patient bloodstream based on the characteristic of resonance signal during the stable concentration portion.

13. The system of claim 12, wherein the ultrasound transducer is further configured to:
correlate a magnitude of the resonance signal during the stable concentration portion to the circulating concentration of the agent in the patient bloodstream.

14. The system of claim 13, wherein the ultrasound transducer is further configured to determine the quantified circulating blood volume based on a volume and concentration) of injected agent divided by the measured circulating concentration of the agent in the patient bloodstream.

15. The system of claim 9, wherein the agent includes a distearoylphosphatidylcholine (DSCP) lipid shell with polyethylene glycol (PEG)-stearate that encloses a heavy gas (such as decafluorobutane) core.

16. The system of claim 1, wherein the agent includes a lipid shell and a heavy gas core.

17. The system of claim 10, wherein the agent has a diameter that is less than 1 micrometer.

18. The system of claim 17, wherein the agent has a neutral charge.

19. The system of claim 1, wherein the ultrasound transducer is further configured to:
transmit an instruction to cause an array of ultrasound transducers to measure the resonance signal of the agent in the patient bloodstream;
compare signal characteristics of reflective signals from multiple ultrasound transducers in the array; and
identify a best available ultrasound transducer in the array based on the signal-to-noise ratio of each of the reflective signals from the multiple ultrasound transducers in the array.

20. The system of claim 19, wherein the compared signal characteristics include a contrast agent signal-to-noise ratio.
